(19) United States
(12) Patent Application Publication
(10) Pub. No.: US 2006/0057236 A1
(43) Pub. Date: Mar. 16, 2006

(54) Process for producing an extract of ivy leaves

(76) Inventors: Frank Runkel, Buseck (DE); Karl-Michael Engelhard, Friedrichsdorf (DE); Georg Maximilian Engelhard, Kronberg (DE)

Correspondence Address: HARNESS, DICKEY & PIERCE, P.L.C., P.O. BOX 828, BLOOMFIELD HILLS, MI, 48303, US

(21) Appl. No.: 11/241,120

(22) Filed: Sep. 30, 2005

Related U.S. Application Data
(63) Continuation of application No. PCT/EP04/03420, filed on Mar. 31, 2004.

(30) Foreign Application Priority Data
Apr. 2, 2003 (DE) 103 15 931.2

(51) Int. Cl. A61K 36/25

(52) U.S. Cl. 424/774

(57) ABSTRACT

A process for producing an extract of ivy leaves using an extracting agent, with the extract comprising α-hederin. In this process, a quantity of dried ivy leaves is first of all comminuted, then fermented by adding water and subsequently extracted by adding the extracting agent; finally, the extract is dried.

### CROSSREFERENCE OF PENDING APPLICATION

This application is a continuation of pending international application PCT/EP2004/003420 filed on Mar. 31, 2004 which designates US and was published in German, and which claims priority of German patent application 103 15 931.2 filed on Apr. 2, 2003.

### BACKGROUND OF THE INVENTION

The present invention relates to a process for producing an extract of ivy leaves using an extracting agent, with the dry extract comprising α-hederin.

Extracts of ivy leaves are nowadays employed successfully for treating respiratory tract diseases, in particular, since the extract exhibits spasmolytic, expectorating and antiobstructive effects. These effects are to be attributed, in particular, to the therapeutically important ingredients of the ivy leaf extracts which belong to the triterpene saponin class. The main saponins in this connection are the bidesmosidic hederacoside C and α-hederin, which is formed from it by ester hydrolysis. Another saponin which has been detected is hederagenin.

Since extracts can be obtained from ivy leaves by a variety of methods, these extracts frequently exhibit different degrees of efficacy. This stems from the fact that the content of the ingredients depends on the particular method used for producing the extract.

On the basis of studies which it has carried out, the applicant has found that α-hederin, in particular, contributes towards bronchospasmolysis. It has been found that α-hederin inhibits the process of internalizing β-adrenergic receptors in a specific manner. Binding of a ligand to the β-adrenergic receptors initially activates the adenylate cyclase system, by way of which the smooth musculature of the bronchial system, for example, is caused to relax as a consequential reaction. Any overshooting of the effect is counteracted, inter alia, by the membrane-located receptors being downregulated by being internalized into the cell.

In this connection, it was possible to show that α-hederin does not attack the β-adrenergic receptor directly but, instead, inhibits internalization of the receptor/ligand into the cell. When the internalization of the receptor/ligand complexes is inhibited, the adenylate cyclase system is activated continuously, thereby intensifying the relaxation of the smooth musculature in the bronchi (spasmolysis).

On the other hand, the substances hederacoside C and hederagenin were unable to inhibit the process of internalization. Accordingly, extracts which have a high content of α-hederin are particularly suitable for being used as pharmaceuticals.

A great variety of methods for producing dry extracts of plant materials are described in the field of pharmacy and of pharmaceutical preparations, in particular.

A method for producing dry extracts of plant materials is disclosed, for example, in DE 101 12 168 A1. Using the method which is disclosed in this publication, it is said to be possible to adjust the content of lipophilic and hydrophilic substances. In this connection, the plant material is subjected to at least two extractions with solvents of differing lipophilicity and the extracts from these extractions are isolated separately. The extracts are dried separately from each other and then mixed in the desired ratio. In this way, it is possible to adjust the content of lipophilic and hydrophilic substances. The method is also said to be suitable for isolating dry extracts of ivy (Hedera helix).

However, a disadvantage of this method is the fact that two separate extractions have to be carried out, thereby making the method as a whole very elaborate.

DE 30 25 223 A1 furthermore discloses a pharmaceutical preparation which is based on ivy extracts and a method for producing them, in which method ivy extracts having hederasaponin C contents of 60 and 90%, respectively, are obtained using acetone and methanol. In order to convert hederasaponin C, or hederacoside C, into α-hederin, and in order, thereby, to obtain an extract which only contains α-hederin, the 90% extract is, in the abovementioned application, hydrolyzed with sodium hydroxide or potassium hydroxide.

However, a disadvantage of this method is the elaborate nature which is occasioned by the additional steps which are required, after hederacoside C has been obtained, for converting the hederacoside, or the hederasaponin, into α-hederin.

G. Wulff “Neuere Entwicklungen auf dem Saponingebiet [Recent developments in the saponin field]”, Deutsche Apotheker Zeitung [German pharmacists' journal], 108 (No. 23), 1968, pp. 797-808, discloses that, if fresh ivy leaves are comminuted and left to stand in water overnight, it is then subsequently only α-hederin which is still found. Enzymes which are present in fresh leaves are held to be responsible for this effect.

However, the pharmaceutical industry employs dried drugs since these latter are stable and easier to handle. Enzymic activities are not to be expected in these dried drugs.

The object of the present invention is therefore to provide a process which can be used to obtain, with few steps and without any great expenditure of time, an extract, in particular a dry extract, in which α-hederin is at least greatly enriched.

### SUMMARY OF THE INVENTION

According to the process which was specified at the outset, the object underlying the invention is achieved by the process comprising the following steps:
a) comminuting a quantity of dried ivy leaves,
b) fermenting by adding water, with hederacoside C being converted into α-hederin,
c) extracting by adding the extracting agent for obtaining an extract, and
d) where appropriate, drying the extract.

Thus, when the process according to the invention is used, it is possible to obtain an extract which comprises α-hederin, as active substance, in enriched form or in the course of whose preparation a major part, or, where appropriate, all, of the hederacoside C which was originally present in the ivy leaves can be converted into α-hederin.

The inventors have shown, in their own experiments, that, when dried ivy leaves are used, the fermentation step ensures a virtually complete conversion of hederacoside C into α-hederin. As a result, no further steps are required, after the extraction, for subjecting the ingredients which have been obtained to any further reaction. On the contrary, precisely as a result of the intercalated fermentation step, the overall process can be carried out more economically, both as regards the time which is gained and materials which are to be employed, with this constituting a major advantage as compared with methods which are described in the prior art.

In the present case, “fermenting” means the breakdown, or the conversion, of ingredients, which were present in an original substance, into other substances when a fermentation medium, for example a liquid, is added to the original substance, with, where appropriate, specific parameters, for example time and temperature, being matched to the fermentation process.

The inventors have found that water is particularly suitable for being used in the fermentation step. Adding the water stimulates the process of the conversion of hederacoside C into α-hederin.

It is therefore possible to use the process according to the invention to produce an extract which has a particularly high content of α-hederin. An extract having a high content of α-hederin is particularly desirable for treating diseases of the respiratory tract since the investigations carried out by the applicant have, of course, established that the responsible factor in this connection is α-hederin.

As mentioned above, it is, in particular, α-hederin, of the saponins which are present in the ivy, which is responsible for the spasmolytic effect in the bronchi. As a result of the high content of α-hederin, the activity of this extract is therefore significantly improved as compared with that of extracts in which a relatively high content of hederacoside C is still present.

In pharmaceutical production, dried drugs have the advantage that they are easier to handle than fresh drugs in regard to stability, inter alia.

In the field of pharmaceutical technology, dried medicinal plants and medicinal plant parts are designated “drugs” by definition. In this connection, such medicinal plants, which are present as “drugs”, are used either in unaltered form or in comminuted form.

It is to be regarded as being surprising to the skilled person that, in the case of dried ivy leaves, as employed in the pharmaceutical industry, a conversion into α-hederin can be achieved simply by adding water.

In the case of DE 30 25 223 A1, which was mentioned at the outset, it was necessary to add alkali metal hydroxides, for the hydrolysis, in order to activate a chemical process.

It is furthermore preferred for the ivy leaves to be comminuted in step a) to ≦5×5 mm, in particular to ≦2×2 mm.

Comminuting the ivy leaves to the abovementioned dimensions is particularly advantageous for exerting an influence on the α-hederin content of the extract which is produced in accordance with the invention. In this connection, it was found that the extraction and fermentation of the ingredients became poorer as the size of the leaf pieces increased.

Preference is furthermore given, in the process according to the invention, to the extracting agent being a mixture of alcohol and water fractions.

Particular preference is given to the alcohol employed being a C₂-C₁₀-alcohol, in particular ethanol.

Ethanol is a proven pharmaceutical solvent and is employed in many different ways as an extracting agent in the field of pharmaceutical technology. However, it is naturally also possible to use other alcohols, for example propanol, isopropanol, etc., that is any alcohols which can be employed as water/alcohol mixtures in the extraction in the context of the pharmaceutical preparation.

Preference is furthermore given to the water/alcohol fractions in the extracting agent being in a ratio (m/m) of from 10/90 to 90/10 and, in particular, to a water/alcohol ratio of 70/30.

In the context of the present invention, a variety of alcohol/water ratios are suitable for successfully implementing the process according to the invention. In this connection, using an extracting agent comprising an alcohol fraction of 30% was found to be particularly suitable.

Preference is furthermore given to the ratio of ivy leaves to extracting agent being from 1:1 to 1:50, in particular 1:12.

In connection with the process according to the invention, the choice of particular quantitative ratios of the ivy leaves to be processed and of the extracting agent can influence the content of α-hederin in the extract. When determining the quantitative ratio, account must be taken of the fact that, on the one hand, hederacoside C has, where appropriate, to be completely converted. On the other hand, the quantity of extracting agent to be employed should be matched to the process such that a successful extraction is ensured while the quantity of extracting agent employed is at an optimally low level. It will be clear to the skilled person that it is possible, for this purpose, to use a variety of quantitative ratios in the process according to the invention.

In one embodiment of the process according to the invention, preference is given to adding a water fraction of the extracting agent in step b) and, in particular, to initially only adding the water fraction from half of the extracting agent.

In this embodiment, the alcohol fraction of the extracting agent and, where appropriate, further extracting agent, are only added after the fermentation. Adding alcohol fractions of the extracting agent after the fermentation with water fractions is advantageous from the process engineering point of view since, accordingly, the quantity of extracting agent to be employed for different batches can in each case be calculated precisely and readily before-hand, and used. The individual volume fractions of the water and the alcohol can then in each case be added separately from each other, or complementing each other, in steps b) and c).

In this connection, it is particularly suitable to add, for the fermentation, the fraction of water contained in half the quantity of the extracting agent, with this making the process even more viable. However, the possibility is not ruled out of also being able to add other quantities of water in order to promote the fermentation, in order, in turn, to influence the process of the conversion of hederacoside C into α-hederin.

Preference is furthermore given to carrying out step b) for a period of from 1 min to 120 min, preferably for a period of 60 min.

By varying the fermentation period, it is possible to exert further control on the α-hederin content of the extract in connection with the process according to the invention.

In this connection, preference is in particular given to the fermentation being carried out at a temperature of between 10 and 40° C., in particular at 30° C.

In another embodiment of the process according to the invention, preference is given to the extraction in step c) being carried out by preswelling and percolation.

Both measures are conventional methods in the prior art and, in particular, in the field of plant-derived pharmaceutical preparations. The steps of preswelling and percolation can be used to achieve an exhaustive extraction of the ivy leaves. Accordingly, after the fermentation with water fractions, the appropriate alcohol fractions are added to the mixture, after which the preswelling is carried out. The remaining fraction of the extracting agent, of which particular fractions have previously been used in the fermentation and the other extraction steps, is preferably added for the percolation.

In this connection, preference is given to the preswelling being carried out for a period of from 1 h to 30 h and, in particular, for a period of 6 h.

In a development of the process according to the invention, preference is given to the drying in step d) being carried out by thin film vaporization and subsequent spray drying.

Both methods are conventional drying methods with thin film vaporization having been found to be a mild evaporation method in connection with preparing pharmaceuticals, just like spray drying, which can, as experience has shown, be used to dry liquid preparations to form pulverulent end products which are distinguished by the fact, in particular, that they can, for example, be readily mixed with water once again to give ready-to-use preparations.

Preference is furthermore given to the process according to the invention being employed for controlling the content of α-hederin and hederacoside in an ivy leaf extract.

In addition, it is possible to use the process according to the invention to regulate the content of α-hederin in the extract in a selective manner. In accordance with this, the process according to the invention can be used either to completely convert hederacoside into α-hederin in an ivy leaf extract or else only to partially convert hederacoside into α-hederin.

Accordingly, it is possible, for example, to initially only subject a given portion of the drug to the fermentation with water and then to add the remaining portion of the drug to the extraction after the fermentation has come to an end. In this way, it is possible to selectively prepare dry extracts in which not all the hederacoside C has been converted into α-hederin.

This furthermore makes it possible to control the content of α-hederin in the extract in a specific manner. This is desirable, for example, when the portion of α-hederin in the extract should not be too high but, rather, a given portion of hederacoside C should still be present.

The invention furthermore relates to an extract of ivy leaves which is produced using the process according to the invention.

Extracts having a high content of α-hederin are advantageous, for example, in connection with treating respiratory tract diseases since, when α-hederin is present in highly concentrated form, it is the most active ingredient in the leaves which is present in highly concentrated form.

The invention furthermore relates to the use of the extract, in particular the dry extract, which has been prepared in accordance with the invention for producing a pharmaceutical and, in particular, for producing a pharmaceutical for treating respiratory tract diseases.

The invention also relates to a pharmaceutical which comprises an extract which has been prepared using the process according to the invention.

In this connection, the pharmaceutical can be present in the form of capsules, tablets, sugar-coated tablets, suppositories, granules, powders, solutions, creams, emulsions, aerosols, ointments and oils. In this connection, oral administration forms are particularly preferred. In this connection, the pharmaceutical can comprise auxiliary substances which are conventionally used in the production of pharmaceuticals. A number of suitable substances can be found, for example, in A. Kibbe, Handbook of Pharmaceutical Excipients, 3rd Ed., 2000, American Pharmaceutical Association and Pharmaceutical Press.

It will be understood that the features which are mentioned above, and those which are still to be explained below, can be used not only in the combination which is in each case specified but also in other combinations, or on their own, without departing from the scope of the present invention.

### BRIEF DESCRIPTION OF THE DRAWING

The invention is described in further detail in connection with the attached figure in which:

FIG. 1a shows a chromatogram of a sample which was not subjected to any fermentation, and

FIG. 1b shows a chromatogram of a sample which was subjected to a fermentation.

### DETAILED DESCRIPTION OF PREFERRED EMBODIMENTS

### EXAMPLES

The extraction method which has previously been carried out in the prior art comprises a multistep extraction method in which 30% ethanol (m/m) is added to the dried drug immediately after it has been chopped. The hederacoside C/α-hederin ratio in the dry extract then corresponds approximately to that in the drug. Analyses of the extracts which have been produced in recent years show a hederacoside C content of from approx. 5 to 20% in the total extract while α-hederin constitutes from about 1 to 5% of the total extract. On the basis of the methods known in the prior art, therefore, a process should be provided by which the conversion of hederacoside C into α-hederin can be included in the extraction method. With this aim in mind, some preliminary experiments were first of all carried out:

1. Extensive and Intensive Comminution of the Fresh, Still Undried Leaves, with Subsequent Drying of the Drug

In order to investigate the influence of the post-harvesting processing on the drug and the subsequent extract, ivy leaves were harvested freshly and carefully by hand. All the leaves were collected at the same time and at the same site and, after harvesting, subjected to different methodological processes. The samples were then analyzed as follows: one sample quantity, which corresponds to about 200 mg of dried drug, was pulverized in a mill and shaken for 1 h together with 30 ml of 45% (v/v) ethanol. After the mixture had been transferred to a chromatography tube, it was percolated with fresh 45% (v/v) ethanol up to a volume of 100 ml. In order to obtain different samples, variations were implemented with regard to the time of comminution and of drying:

Sample 1: The fresh leaves were cut into pieces of 3×3 mm in size and only dried after having been stored for 12 hours.

Sample 2: The fresh leaves were cut into pieces of 3×3 mm in size and then immediately dried.

Sample 3: The uncut leaves were dried at 50° C. after harvesting and only comminuted into pieces of approx. 3×3 mm in size shortly prior to the extraction process.

The following Table 1 records the chromatographic investigations with regard to the content of α-hederin and hederacoside C:

TABLE 1

| | | Hederacoside C | Total saponins calculated |
|---|---|---|---|
| | α-Hederin | (%) | as hederacoside C |
| Sample 1 | 1.25 | 6.05 | 8.08 |
| Sample 2 | 1.40 | 5.45 | 7.73 |
| Sample 3 | 1.13 | 5.75 | 7.59 |

The results are comparable with each other and do not differ significantly from each other even though there appears to be a slight displacement of the hederacoside C/α-hederin ratio in favor of the α-hederin in the case of the leaves which are cut in the fresh state.

In further investigations, variations were carried out with regard to the degree to which the ivy leaves were comminuted and with regard to the time at which the content was determined:

Sample 1: The fresh leaves were triturated finely and immediately extracted with 45% (v/v) ethanol.

Sample 2: The fresh leaves were triturated finely and extracted with 45% (v/v) ethanol after a delay of 45 min.

Sample 3: The uncut leaves were dried at 50° C. after harvesting and comminuted shortly prior to the extraction process.

The results of the chromatographic investigations with regard to the α-hederin and hederacoside C content are recorded in Table 2 below:

TABLE 2

| | | | Total saponins |
|---|---|---|---|
| | | Hederacoside C | calculated as |
| | α-Hederin | (%) | hederacoside C |
| Sample 1 | 3.74 | 0.37 | 6.45 |
| Sample 2 | 3.17 | 0.19 | 5.34 |
| Sample 3 | 1.13 | 5.75 | 7.59 |

As can be seen from the table, the intensive comminution converted almost all the hederacoside C into α-hederin.

2. Fermentation of the Dried and Comminuted Drug Prior to the Extraction

In order to destroy the integrity of the cells, the process of the conversion of hederacoside C into α-hederin was stimulated by extensive comminution and adding water. This was verified by the following experiment:

Whole, dried ivy leaves having a previously determined content of 7.94% hederacoside C and 0.59% α-hederin were treated as follows:

Sample 1: 20 ml of water are added to 200 mg of the extensively comminuted leaves. After the water has been evaporated off in vacuo, the mixture is extracted as usual with 45% (v/v) ethanol.

Sample 2: The drug is treated as Sample 1 but 30% (m/m) ethanol is used in place of the water.

The chromatographic results with regard to the content of α-hederin and hederacoside C are recorded in Table 3 below:

TABLE 3

| | | | Total saponins |
|---|---|---|---|
| | | Hederacoside C | calculated as |
| | α-Hederin | (%) | hederacoside C |
| Sample 1 | 4.40 | 0 | 7.15 |
| Sample 2 | 1.12 | 6.51 | 8.33 |
| Start | 0.59 | 7.94 | 8.90 |

As can be seen from the data, the hederacoside C was completely converted into α-hederin by adding water to the comminuted drug.

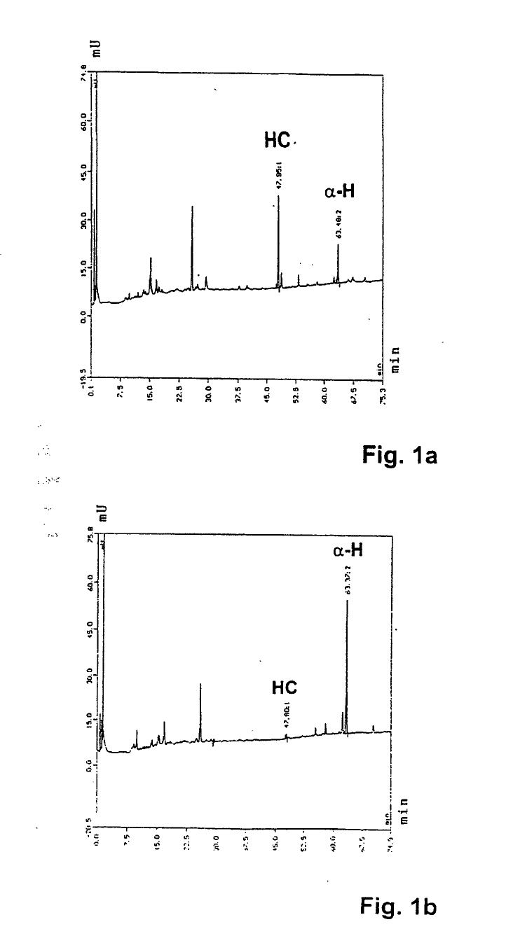

FIG. 1 shows the chromatograms for Sample 1 (FIG. 1b) and Sample 2 (FIG. 1a) which were obtained by HPLC. In the figures, the peak which was obtained for the substance hederacoside C is labeled “HC” while the peak obtained for α-hederin is in each case labeled “α-H”. As can be seen by comparing the peaks in FIGS. 1a and 1b, the content of hederacoside C is almost completely reduced at the expense of the α-hederin, with this demonstrating that all the hederacoside C was converted.

While the fermentation in 30% (m/m) ethanol also resulted in a breakdown of the hederacoside C, the extent of this breakdown was markedly lower.

In other investigations, it was shown that the degree of comminution of the drug and the quantity of water supplied are of crucial importance for the extent of the conversion: while extraction experiments using approx. 5×5 mm-sized leaf fragments, which were fermented as described in water, showed an enrichment of the α-hederin at the expense of the hederacoside C, the conversion was incomplete owing to many of the cells being intact. The experiment of only lightly spraying finely comminuted leaves with water also resulted in the conversion of the hederacoside C being incomplete.

The following variants of an extraction method were carried out in order to convert the abovementioned previously obtained findings with regard to the conversion of hederacoside C into α-hederin into a successful extraction protocol:

Extract 1: 3.0 g of comminuted ivy leaves (5×5 mm) were extracted with 12-times the quantity of 30% (m/m) ethanol (normal extraction).

Extract 2: 3.0 g of comminuted ivy leaves (5×5 mm) were covered with water in a chromatography column. After 1 h, the water was drained off through a draw-off tap, with the leaves then being extracted with 12-times the quantity of 30% (m/m) ethanol.

Extract 3: The water from 12 parts of extracting agent (30% (m/m) ethanol) was added to 3.0 g of comminuted ivy leaves (5×5 mm). After a fermentation time of 1 h, the ethanol fraction from 12 parts of extracting agent was added in order to extract the leaves.

The chromatographic analyses gave the results shown in Table 4 below:

TABLE 4

| | | | Total saponins |
|---|---|---|---|
| | | Hederacoside C | calculated as |
| | α-Hederin | (%) | hederacoside C |
| Extract 1 | 0.48 | 19.80 | 20.58 |
| Extract 2 | 4.11 | 1.38 | 8.06 |
| Extract 3 | 8.38 | 5.88 | 19.51 |

Since, in these extractions, the drug was only comminuted to 5×5 mm, the conversion was not complete. It can be clearly seen from the table that draining off the fermentation water (Extract 2) led to loss of the ingredients.

Combining the above-described preliminary experiments resulted in the development of another extraction process, which can be used to control the content of α-hederin in a dry ivy leaf extract.

3. Extraction Process for Producing Dry Ivy Leaf Extracts having a Controllable Content of α-hederin

After using quality assessment to test the quality and release, a portion of the drug (DAC ivy leaves) was extensively comminuted in a mill, with a protective screening guaranteeing that the size of the fragments was at most 2×2 mm. In addition, the screened material was examined optically for larger particles and impurities.

The water fraction from 6 parts of extracting agent (30% (m/m) ethanol) was added to the comminuted sample. This mixture was fermented at 30° C. for 60 min while occasionally mixing/stirring.

The 96% ethanol fraction from 6 parts of the extracting agent was then added and the mixture was homogenized by stirring.

After a 6-hour preswelling phase, the eluate was separated off and the drug which remained behind was percolated with the remaining 6 parts of the extracting agent.

The combined eluates were filtered once again, in order to exclude small drug particles, and homogenized before they were dried to a thick paste by means of thin film vaporization at 55° C. and 150 mbar. This paste was homogenized and then dried by spray drying at from 45 to 60° C. to give the dried ivy leaf extract.

In order to examine this production process, the following extracts were prepared: proceeding from ivy leaves having a content of 3.91% hederacoside C and 0.20% α-hederin, an extract was prepared on the one hand in accordance with the conventional method (comminuting the dried sample with subsequent direct addition of 30% (m/m) ethanol, and extraction) and on the other hand in accordance with the novel method. The chromatographic results with regard to the content of α-hederin and hederacoside are recorded in Table 5 below:

TABLE 5

| | | | Total saponins |
|---|---|---|---|
| | α-Hederin | Hederacoside C | calculated as |
| | (%) | (%) | hederacoside C |
| Conventional | 0.53 | 8.68 | 9.54 |
| Novel | 4.74 | 0 | 7.71 |

As can be seen from the table, the novel extraction process was able to completely convert the hederacoside C which was present in the leaves into α-hederin.

Since the total saponin content, calculated as hederacoside C, is also of the same order of size, it is possible, knowing the corresponding saponin concentration in the drug and taking into account the enrichment factor of from approx. 2 to 3, to estimate the final content of α-hederin in the extract.

In order, therefore, only to convert a part of the hederacoside C present in the drug into α-hederin, it is possible only to subject a defined fraction of the drug to the fermentation with water while keeping all the other parameters constant. The remaining drug, and the ethanol, are then added for the 6-hour preswelling after the 60-minute fermentation has come to an end.

It is therefore possible to use the process according to the invention to markedly alter the ingredient spectrum of a dry ivy leaf extract, without using any large input of time and resources, by introducing a fermentation. Not least as confirmed by the large number of publications on the activity of α-hederin, the selective influencing of the α-hederin content in a dry extract, or pharmaceutical, has a positive effect on the activity of the extract.

What is claimed is:

1. A process for producing an extract of ivy leaves by using an extracting agent for obtaining an extract comprising α-hederin, said method comprising the following steps:
a) comminuting a quantity of dried ivy leaves,
b) fermenting by adding water, with hederacoside C being converted into α-hederin,
c) extracting by adding an extracting agent for obtaining an extract, and
d) where appropriate, drying said extract.

2. The process of claim 1, wherein said ivy leaves are comminuted in step a) to ≦5×5 mm.

3. The process of claim 2, wherein said ivy leaves are comminuted to ≦2×2 mm.

4. The process of claim 1, wherein a mixture of alcohol and water is employed as said extracting agent.

5. The process of claim 4, wherein said alcohol employed in said extracting agent is a C₂-C₁₀-alcohol.

6. The process of claim 5, wherein said alcohol is 96% ethanol.

7. The process of claim 1, wherein said extracting agent is a water/alcohol mixture in a ratio (m/m) of from 10/90 to 90/10.

8. The process of claim 7, wherein said ratio is 70/30.

9. The process of claim 1, wherein a ratio of ivy leaves to said extracting agent is from 1:1 to 1:50.

10. The process of claim 9, wherein said ratio of ivy leaves to said extracting agent is 1:12.

11. The process of claim 1, wherein a water fraction of said extracting agent is employed in step b).

12. The process of claim 11, wherein a half of a total quantity of said extracting agent is added in step b) as said water fraction.

13. The process of claim 1, wherein step b) is carried out for a period of from 1 min to 120 min.

14. The process of claim 13, wherein step b) is carried out for a period of 60 min.

15. The process of claim 1, wherein step b) is carried out at a temperature of from 10 to 40° C.

16. The process of claim 15, wherein step b) is carried out at a temperature of 30° C.

17. The process of claim 1, wherein said extraction in step c) is carried out by a preswelling and a percolation of said ivy leafs.

18. The process as claimed in claim 17, wherein said preswelling is carried out for a period of from 1 h to 30 h.

19. The process of claim 18, wherein said preswelling is carried out for a period of 6 h.

20. The process of claim 1, wherein said drying in step d) is carried out by a thin film vaporization and a subsequent spray drying.

21. An extract of ivy leaves, obtained by a method of claim 1.

22. A pharmaceutical comprising an extract as obtained by the method of claim 1.

23. The pharmaceutical of claim 22 for treating respiratory tract diseases.
